(19) United States
(12) Patent Application Publication
(10) Pub. No.: US 2007/0243572 A1
(43) Pub. Date: Oct. 18, 2007

(54) Interacting Microhabitat Array and Uses Thereof

(76) Inventors: Juan Keymer, Princeton, NJ (US); Peter Galajda, Princeton, NJ (US); Robert H. Austin, Princeton, NJ (US)

Correspondence Address: CONNOLLY BOVE LODGE & HUTZ, LLP, P O BOX 2207, WILMINGTON, DE, 19899, US

(21) Appl. No.: 11/623,595

(22) Filed: Jan. 16, 2007

Related U.S. Application Data
(60) Provisional application No. 60/759,607, filed on Jan. 17, 2006.
(60) Provisional application No. 60/849,076, filed on Oct. 3, 2006.

(51) Int. Cl. C12Q 1/02; C12N 1/21

(52) U.S. Cl. 435/29; 435/243

(57) ABSTRACT

The invention is directed to an interacting microhabitat array for microorganisms having more than one microhabitat in a substrate in which at least two microhabitats are connected in series by at least one corridor. The corridor is of sufficient size to allow the microorganism to move between microhabitats in a restricted manner. The invention is also directed to uses of the device for screening for method of modulating biofilms and identifying drug candidates.

### PRIORITY

This application claims priority to U.S. Provisional Application 60/759,607 filed on Jan. 17, 2006 and U.S. Provisional Application 60/849,076 filed on Oct. 3, 2006, both of which are incorporated herein by reference in their entireties.

### STATEMENT OF GOVERNMENT SUPPORT

The government may have rights to this invention under the following grant: Air Force Office of Scientific Research grant FA 9550-05-01-0365.

### BACKGROUND OF THE DISCLOSURE

1. Field of the Invention

The invention relates generally to arrays of microscale or nanoscale habitats for microorganisms. The arrays may be used for studying the evolutionary biology and ecology of the microorganisms and for evaluating the effect of outside stimuli or agents on the populations of microorganisms. For example, the arrays can be used to model bacterial biofilms and can be used to screen drug candidates for antibacterial effect.

2. Background

In nature, habitats are patchy, aggregating at several scales, thereby generating a landscape of discrete habitats. Existing devices for culturing microorganisms do not reflect natural patchy environments, but rather attempt to provide uniform conditions for growth. In general, such devices do not permit development of a metapopulation or “population of populations.” Hanski I A, Gilpin M E, eds (1997) Metapopulation Biology: Ecology, Genetics, and Evolution (Elsevier, Burlington, Mass.).

Some microscale bioreactors have been described. Jensen et al. (US2004/0077075) discloses a variety of microscale bioreactors and bioreactor arrays for use in culturing cells. Balagadde et al. (US 2005/0164376) discloses a chemostat having a growth chamber having a plurality of compartments. Zhang et al. (US2006/0199260) discloses a microscale bioreactor and bioreactor array for use in culturing cells. Groisman et al. discloses a microfluidic chemostat suitable for use with bacteria and yeast cells. Groisman et al. (2005) Nat Methods 2:685-689.

In general, known microfabricated chemostats oust as the macroscopic ones) do not allow for the emergence of a metapopulation, that is, a spatially distributed network of parallel populations adapted to different local conditions but weakly coupled with one another. In part, this results from the uniform environments that existing chemostats and bioreactors provide. Because chemostats lack spatial structure, they do not allow organisms to search out different niches in a spatially heterogeneous habitat. Unlike the artificial scheme of existing model systems, natural habitats are heterogeneous.

Bacteria and other microorganisms self-organize into sophisticated dynamic assemblages. Escherichia coli individuals are known to exhibit complex patterns of motility. Budrene E O, Berg H C (1991) Nature 349:630-633. Individual bacteria are known to associate even further into very complex communities (biofilms) that resemble a human metropolis (Watnick et al.) in which microbes communicate with each other (Basler) and work together toward common goals (Greenberg), exploiting what is called niche complementarity (Kinzig et al.). Watnick et al. (2000) J Bacteriol 182:2675-2679; Bassier B (2002) Cell 17:421-424; Greenberg P (2003) Nature 424:134; and Kinzig et al. (2001) The Functional Consequences of Biodiversity (Princeton Univ. Press, Princeton, N.J. US). It would be useful for studying bacterial biofilms to have a laboratory model that allows for the controlled generation of biofilms. Specifically, it would be useful to have a model for generating biofilms to use to screen for antibiotic agents or other therapeutic agents whose administration to the mammalian body is likely to require contact with or passing through a biofilm to reach the tissue or cells where it can exert a therapeutic effect.

It would also be useful to have a model that allows for the directed evolution of microorganisms having desired properties. For example, some microorganisms may be used industrially to produce useful products. Such useful microorganisms include those that have developed a photochemistry which can generate H₂, which can be used as an energy source. H₂ production from photobiology has the enormous advantage over H₂ production from fermentation in that the intermediate step of biomass production is eliminated. Cyanobacteria and certain algae can use light to oxidize water, generating H₂ directly from water.

The present invention concerns an Interacting Microhabitat Array (IMA) and uses of the IMA such as, for example, studying evolution of microorganisms and directing evolution of microorganisms, including H₂ generating microorganisms. Both algae (which are complex eukaryotic organisms) and photobacteria (somewhat simpler prokaryotic organisms) can produce H₂ directly from sunlight and evolution of these microorganisms is believed to be observed through growth in the microhabitat arrays of the invention.

### SUMMARY OF THE INVENTION

The present invention concerns an interacting microhabitat array in which at least two microhabitats are connected by a corridor, and methods of using the device.

The interacting microhabitat array is for microorganisms and comprises a plurality of microhabitats in a substrate. At least two microhabitats are connected in series by at least one corridor. The corridor is of sufficient size to allow the microorganism to move between microhabitats. The microorganism's movement is not completely obstructed, but is partially restricted such that different populations of the microorganism are able to develop on the array such that the array represents a metapopulation. In one aspect, the microorganism is a single-celled organism.

The invention also concerns a method of evaluating population colonizations and extinctions in a metapopulation of microorganisms comprising introducing at least one species of microorganism into an interacting microhabitat array as described above, providing nutrients to the microhabitats, removing waste from the microhabitats, and detecting population changes during a plurality of generations across the interacting microhabitat array.

The inventors also disclose a method of screening for an agent capable of modulating biofilms comprising: a) culturing a species of microorganism capable of forming a biofilm in the interacting microhabitat array of the invention such that at least one biofilm is formed, (b) adding a test agent to the culture, and (c) detecting whether a change occurs in the biofilm in the presence of the test agent.

The disclosure is also drawn to a method of screening for an agent capable of modulating a microorganism population comprising: a) culturing at least one species of microorganism in an interacting microhabitat array such that metapopulations of the microorganism are formed, (b) adding a test agent to the microhabitat array, and (c) detecting whether a change occurs in the metapopulations of the microorganism in the presence of the test agent.

### BRIEF DESCRIPTION OF THE FIGURES

FIG. 1 is a schematic top plan view of a 1D IMA having two cell input/outlet ports which communicate with the microhabitats and four input/outlet ports connecting to two fluid/feeding channels. The microhabitats are connected by corridors. The microhabitats are connected to the fluid/feeding channels by nanoslits (not shown).

FIG. 2 is a schematic top plan view of a single microhabitat with two adjacent fluid or feeding channels that are connected to the microhabitat by nanoslits.

FIG. 3 is a schematic fragmental perspective view of a single microhabitat with one adjacent fluid/feeding channel connected to the microhabitat by two nanoslits.

FIG. 4 is a schematic of a 2D IMA. Dimensions are shown in microns (μm). FIG. 4A illustrates a microhabitat suitable for a 2D IMA, having a corridor to a neighboring microhabitat. FIG. 4B illustrates a group of nine microhabitats. The arrows illustrate movement of cells and/or nutrients and/or wastes from the microhabitat of FIG. 4A to an adjacent microhabitat. FIG. 4C illustrates a 2D IMA biochip having 160 microhabitats, two fluid/feeding inlet/outlet ports, and two cell loading/unloading ports.

FIG. 5 is a schematic fragmental perspective view of a 2D IMA having a fluid and/or feeding channel, showing all or part of four adjacent microhabitats.

FIG. 6 represents time-sequence images of bacterial populations in a coupled set of linear microhabitats. FIG. 6A shows bacterial densities for 10 coupled microhabitats (on the y-axis). FIG. 6B shows a false color/tone graphic representation showing population density. As the bacteria grow they jump between different microhabitats in a collective and dynamic manner.

FIG. 7 illustrates use of UV laser irradiation to destroy undesired populations and create a destroyed microhabitat to modulate metapopulations in dynamic landscapes. An empty microhabitat can be colonized to form an occupied microhabitat. Monte Carlo sampling of microhabitats and the destructive power of the UV laser is used to implement a dynamic landscape of microhabitats.

FIG. 8A is a photograph of a 1D IMA having two feeding channels, with C. reinhardtii populating some microhabitats. FIG. 8B is a detail of FIG. 8A showing the nanoslit organization, the microorganism, and gas produced by the microorganisms.

FIG. 9 represents a time course of growth of a microorganism in a flat landscape, including lag phase, exponential phase, and stationary phase. FIG. 9A illustrates a time course in an 85 microhabitat IMA, with a detail shown. “MHP” indicates microhabitats. FIG. 9B is a chart of population density. Exponential phase is demarcated by vertical dashed lines.

### DETAILED DESCRIPTION

Definitions

The measurements of length used herein include centimeter (cm, 10⁻² meter), millimeter (mm, 10⁻³ meter), micrometer (10⁻⁸ meter) usually termed “micron”, and nanometer (nm, 10⁻⁹ meter).

“Microhabitat” as used herein is a space having a defined volume which is suitable for the growth and/or maintenance of a population of a microorganism. The volume of a microhabitat is envisioned as between about 1.0 cm³ and 1.0 microns (10⁻⁶ m³). In one aspect the volume of a microhabitat is less than about 1 millimeter³ (mm³). In other embodiments, the volume is between about 1 cm³ and 1.0 mm³, or between about 1.0 mm³ and 1.0 micron³ (10⁻⁶ m³). Alternatively, the volume can be less than about 0.1 mm³ or less than about 0.01 mm³. In a particular embodiment, microhabitat encompasses spaces having a volume of less than about 0.001 mm³, i.e. less than about 1×10⁶ microns³.

An “interacting microhabitat array” or IMA is a plurality of microhabitats in which each microhabitat is connected to at least one other microhabitat with at least one corridor such that a microorganism of interest can move from one habitat to another habitat, allowing local populations to develop on different regions of the array.

The movement of the microorganism should be restricted, but not completely obstructed. The movement can be restricted by about two-fold to about ten-fold over the mobility in free solution. Alternatively, the movement can be restricted by about ten-fold to about 100-fold over the mobility in free solution. In another embodiment, the rate of movement between microhabitats is from about 0.01 microns/sec to about 100 microns/sec. Functionally, this limited mobility allows for controlling the communication between different microhabitats so that metapopulations are allowed to develop on the array.

An interacting microhabitat array has provisions for loading and removing microorganisms. Particular embodiments of an interacting microhabitat array may have detectors for measuring changes in the microhabitats, and channels for supplying or removing fluids, solutes, or nutrients. A zero-dimensional (0D) array has one or more microhabitats, but there is no provision for transfer of microorganisms between microhabitats. A one-dimensional array (1D) has at least two microhabitats that are connected by a corridor that permits transfer of microorganisms. One-dimensional arrays can have a plurality of microhabitats arranged in a linear fashion, an undulating fashion, or any other suitable arrangement. A two-dimensional array (2D) has at least four microhabitats and has at least one microhabitat that is connected to at least three other microhabitats by corridors that permit migration of microorganisms. Two-dimensional arrays can have a plurality of microhabitats arranged in a rectilinear fashion, a hexagonal fashion, or any other suitable arrangement.

An IMA can be fabricated from any suitable material or combination of materials, including, but not limited to glass, silicon, silicon rubber, plastic, and derivatives thereof, and combinations of these materials. For certain embodiments, such as for culturing photosynthetic bacteria or genetically modified microorganisms that express a visible reporter protein, the preferred microhabitat is optically transparent. The substrate for the IMA may also contain materials to allow for monitoring of the evolution of the microorganisms, such as an extra nanosensor element in the substrate that monitors the physiological or the genetic state of the microorganisms. See, e.g., Hood, L. et al. (2004) Science 306: 640-643.

The IMA for example can be fabricated by etching in a silicon wafer. It can also be prepared from polydimethyl-siloxane (PDMS) following the general procedures used to microfabricate a maze in S. Park et al., “Influence of Topology on Bacterial Social Interaction,” PNAS (2003): 100: 13910-13915. Other useful methods of fabrication that can be adapted to make the IMA of the present invention are disclosed in Groisman et al. Groisman A, Lobo C, Cho H, Campbell J K, Dufour Y S, Stevens A M, Levchenko A (2005) Nat Methods 2:685-689. Others methods of fabricating substrates on the micro- or nanoscale are well known to those in the art.

“Nanoslit” is a passageway suitable for fluid exchange between a microhabitat and a reservoir, such as a fluid channel for providing nutrients and removing waste. A “horizontal” nanoslit may be nanoscale in depth but can be very wide and very long. Such a nanoslit corresponds to a thin etched sheet. A “vertical” nanoslit may be nanoscale in width but very wide and long. The size of a nanoslit is important as the function of a nanoslit is to prevent passage of the microorganism of interest while permitting exchange of fluids. In some embodiments this function is achieved by making the smallest cross-sectional dimension of the nanoslit smaller than the smallest dimension of the microorganism of interest.

The microorganism of interest can be any microorganism having a useful property which is small enough to travel through the connecting corridors and inhabit a microhabitat. “Microorganism” is an organism that is small, motile, and reproduces fairly rapidly. “Microorganism” as used herein includes multicellular organisms such as nemotodes and specifically Caenorhabditis elegans. The microorganism also may be a single-celled organism. The microorganism can be a member of the Bacteria, Archae, Protists, or Fungi families, or other eukaryotic cells, e.g. immortalized cells in culture. Indeed, the microorganism can be Escherichia sp., Chlamydomonas sp., Staphylococcus sp., Streptococcus sp., Lactobacillus sp., or Spirochete sp., or a combination thereof. In particular applications, which will be clear from the context, the microorganism can be a virus or a mycoplasma. The microorganism can also be a combination of microorganisms, for example a bacteriophage virus and a bacterium. In a particular embodiment, the microorganism can be a virus alone, for example the RNA virus Q\Beta replicase.

The populations in a microhabitat are described by the following notation: state “−1” corresponds to microhabitats in which the population has been destroyed; state “0” are empty microhabitats; and state “+1” are microhabitats having a population of microorganisms.

One Dimensional Array Without or With Feeding Channels

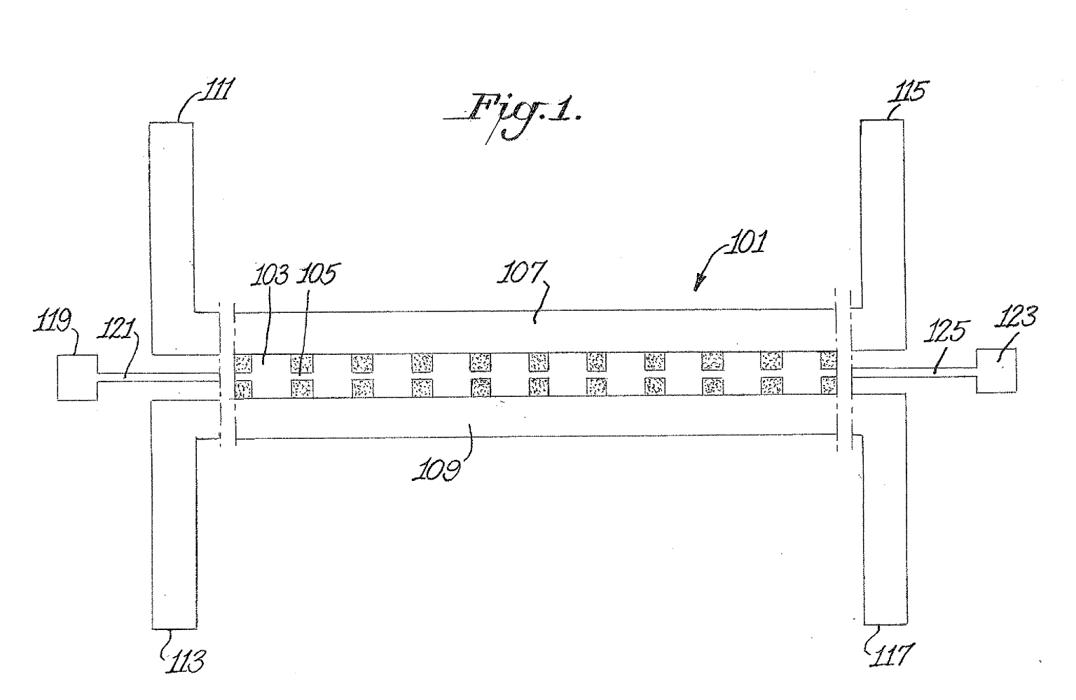

FIG. 1 illustrates a one dimensional array of microhabitats in which feeding channels are present alongside the microhabitats. In an alternative embodiment, the feeding channels are omitted or partially omitted. In this latter embodiment, the microhabitats are loaded with necessary nutrients at or before the time that the microorganism is added.

In a model of the 1-dimensional technology, the design has 0.2 micron (200 nm) deep nanoslits which can introduce and remove nutrients to the microhabitat without removing the bacteria, because the microorganisms cannot penetrate a 0.2 micron wide gap. The basic design is coupled microhabitats, and transverse to this line is an array of nanoslits which are etched down 0.2 microns but are 20 microns wide. Adjacent microhabitats are connected by corridors that permit restricted interchange between microhabitats. The transverse set of nanoslits allows the user to (1) confine the bacteria to the coupled array of microhabitats, (2) feed the bacteria as in a standard habitat, and (3) probe the output of the microhabitat arrays on an individual scale. One can spatially separate the probing chemistry from the colony itself to avoid interference. Nanoslits can include support structures, e.g. posts, to prevent collapse of the nanoslits. Alternatively, the nanoslits may be a forest of closely-spaced posts that prevent escape of the organism. In one embodiment, the device (101) is created by etching a Si wafer to form a row of microfabricated 100 microns×100 microns×30 microns microhabitats (103) that are weakly linked to each other by a corridor (105) or series of corridors and to a source of fluid and/or nutrients (107 and 109). The fluid and/or nutrients is/are provided through input ports (111) and (113) and can leave the device through exit ports (115) and (117). In another embodiment, some or all of the input and exit ports can be interchanged. Cells and/or fluid and/or nutrients can be provided through interface chamber (119) through channel (121) and overflow can exit through interface chamber (123) through channel (125). In this example, the array comprises 85 interconnected microhabitats (103). The top of the array is enclosed by glass and sealed with silicon rubber.

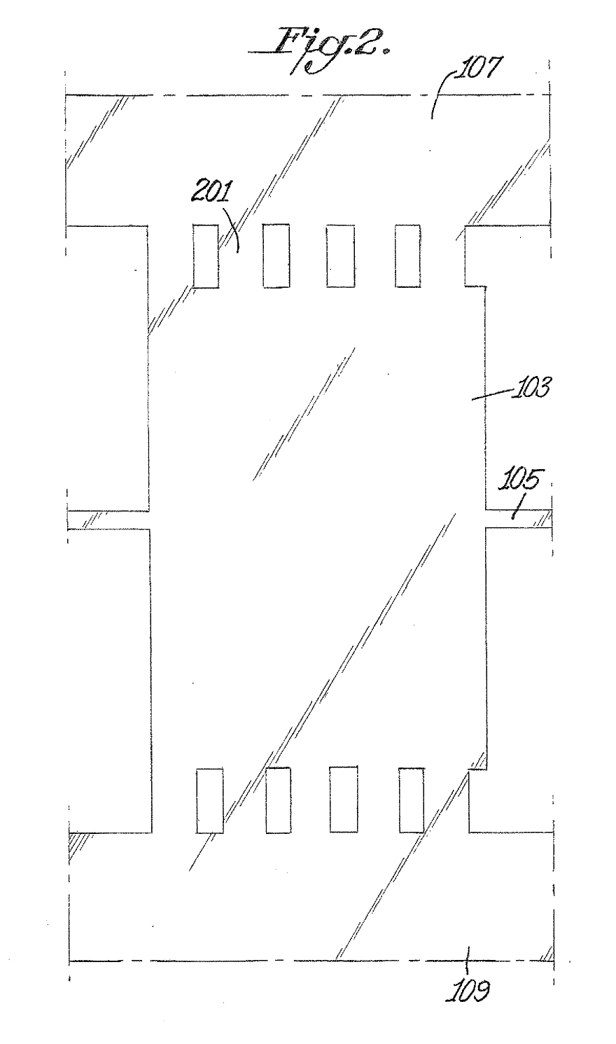

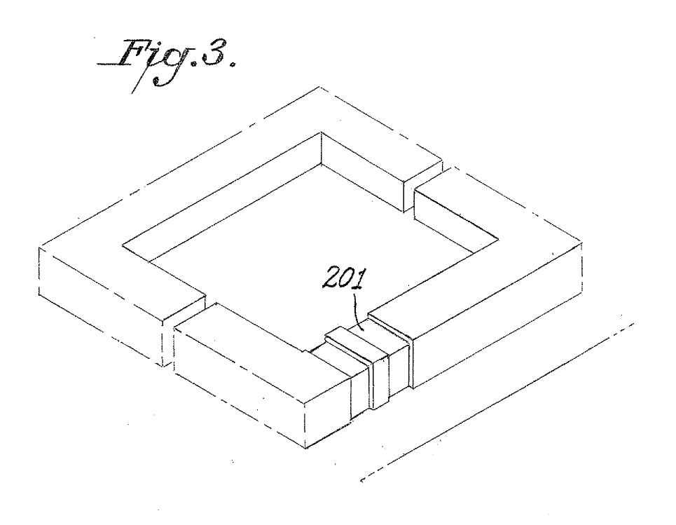

Turning to FIG. 2, fluid and/or nutrients is/are available to the microhabitats (103) from the channels (107 and/or 109) by nanoslits (201). Importantly, either the width or the height or both of the nanoslits is less than the smallest diameter of the microorganism of interest, which prevents the microorganisms from moving into the fluid or nutrient channel. The potential rate of fluid exchange can be increased by increasing the number of nanoslits or their cross-section. FIG. 3 illustrates nanoslits of 0.2 microns in depth, suitable for use with E. coli. The number of nanoslits can be varied from zero to any useful upper number. Indeed, each microhabitat can have a different number of nanoslits. This would allow for heterogeneity in the array and favor the development of discrete populations within the array. Ten nanoslits is a useful value for a high of level nutrient access. In another alternative, a single full-width nanoslit can be used. One or more fluid channels can be accessed by nanoslits. Thus, FIG. 2 shows two fluid channels each accessed by five nanoslits. FIG. 3 shows one fluid channel accessed by two nanoslits.

In one embodiment, the microhabitats are weakly linked together by 50 micron-long, 5 micron-wide, and 30 micron-deep corridors (105) connecting adjacent microhabitats. FIG. 1. The narrow dimension was chosen to permit movement of E. coli from one chamber to the next, but to inhibit the tumbling motion that characterizes E. coli when changing direction. Thus, reversal of direction is discouraged. An alternative design having stronger coupling uses wider corridors that permit movement of more microorganisms and ready reversal of microorganism motion. In one embodiment, the final (1D) device consisted of a chain of 85 microhabitats. FIG. 1. The array was seeded from one end by bacteria from a larger “interface chamber” (119) so that an average of at least one bacterium was put into each chamber. In another embodiment, at least one microorganism is seeded into the entire IMA.

Two-Dimensional Array Without Feeding Channels

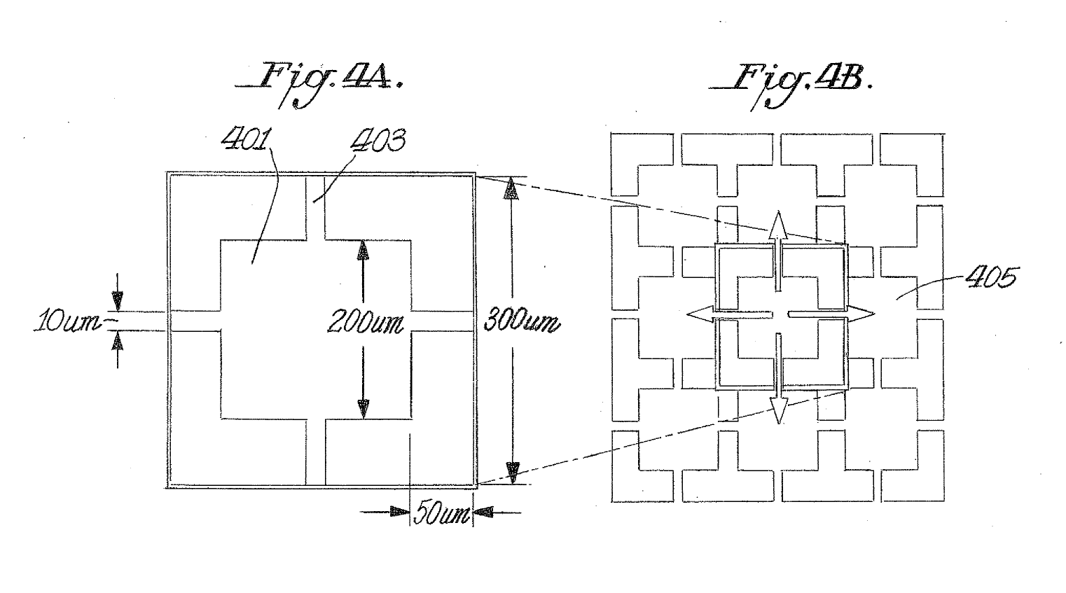

An IMA can be two-dimensional in which a microhabitat can connect to at least three other microhabitats by corridors. See FIG. 4. Panel 4A illustrates a top plan view of a microhabitat (401) having four connecting corridors (e.g. 403). Panel 4B shows a 2D array of nine microhabitats, including 405. The arrows indicate potential movement of organisms from one microhabitat to adjacent microhabitats. Panel 4C shows a biochip (417) having 160 microhabitats arranged in a 40×40 pattern, ports (413) and (415) for loading and unloading the microorganisms, and two ports (409, 411) for nutrient fluid inlet and outlet. The flow from a nutrient fluid port is optionally baffled by a forest of columns (407) that distribute the fluid to all corridors.

Two-Dimensional Array With Feeding Channels

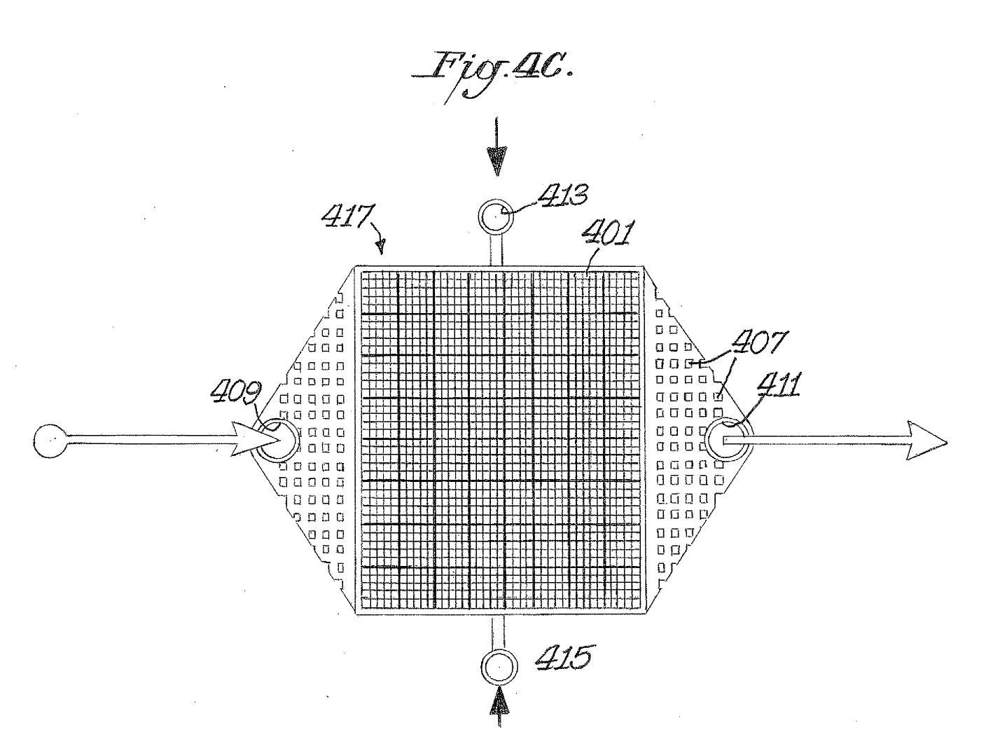

A two-dimensional IMA can also have feeding channels. See FIG. 5. In FIG. 5, 501, 503, 505, 507, 509, and 511 refer to individual microhabitats arranged in a 2D array. Connecting corridors 513, 515, 517, 519, 521, 523, and 525 connect microhabitats with neighboring microhabitats. A fluid/feeding channel 527 is shown connected to microhabitat 501 by nanoslit 529 and to microhabitat 503 by nanoslit 531. To prevent movement of microorganisms from the connecting corridor 529 to the fluid/feeding channel 527, a pair of nanoslits 533 and 535 separates the corridor from the channel, yet permits movement of fluid. Moreover, multiple nanoslits can be used to increase fluid flow.

When desired, a fluid supply channel is provided with access to at least one microhabitat in the array. The fluid supply channel can supply nutrients and remove waste. The channel can be connected to the microhabitat by at least one nanoslit, wherein the nanoslit is of smaller cross-section than the corridor and the nanoslit is of a size such that the microorganism cannot diffuse through it. The IMA can further comprise a second fluid supply channel and a second nanoslit connecting the second fluid supply channel to the microhabitat, whereby cross-flow of fluid is optionally achieved. The IMA can further comprise a detector that can be used to monitor the growth, function and evolution of the microorganisms in the array. The detector can be an optical detector such as a photomultiplier tube or CCD camera. The detector can comprise a sensor element capable of sensing pH or chemicals, for example H₂. The detector can be a nano-scale sensor. In one aspect, a microhabitat has a port to allow remote sensing of the contents. In certain uses, it is desired to destroy microorganism populations, such as those that have evolved in an undesirable direction. For such uses, the IMA may be combined with a device for targeted destruction of populations of certain microhabitats, such as a laser beam. Destruction can also be caused by targeted supply of a toxin to the microhabitats with the undesirable microorganisms.

The microhabitat of the IMA can be wider than the corridor. In one aspect, the corridor is about 5 microns in its smallest dimension. In one aspect, at least one corridor has a length between about 20 microns and about 1000 microns. The IMA can have at least one microhabitat between about 20 to about 500 microns in top width. In another aspect, the microhabitat of the IMA has a top width between about 200 microns to about 2000 microns. The IMA can have at least one microhabitat between about 6 and about 150 microns in side width. In another aspect, at least one microhabitat of the IMA has a side width between about 80 to about 320 microns. In one aspect, the microhabitats in the array are each of approximately the same volume.

The IMA may have any number of microhabitats connected to form the array. In one aspect, the IMA has less than five, 5-10, 10-20, 10-50, 50-100, 50-250, 250-800, or 200 or more microhabitats. In another aspect, the IMA comprises about 85 or about 160 microhabitats.

The IMA can contain at least one microorganism. In one aspect, the microorganism has been genetically modified to express a reporter or fluorescent protein, such that the growth and extinction of population colonizations in a metapopulation of microorganisms can be readily identified. In one particular aspect, the fluorescent protein is a “green fluorescent protein” (GFP), but GFP-like proteins and also proteins which require a co-factor to fluoresce, for example luciferase are also envisioned. The GFP-like protein from the sea pansy (Renilla reniformis) has a single major excitation peak at 498 nm. Modified forms of GFP can also be used.

In one aspect, the method of the disclosure can further comprise at least one second microorganism.

The IMA can be used in screening methods. For example, a model biofilm can be developed in an IMA. The method of screening for an agent capable of modulating biofilms can involve detecting a change by observing an increase in the amount of the species of microorganism forming the biofilm, a decrease in the amount of the species of microorganism forming the biofilm, an increase in the ratio of planktonic cells to cells in the biofilm, a decrease in the ratio of planktonic cells to cells in the biofilm, an increase in the size of the biofilm, or a decrease in the size of the biofilm. In one aspect, the agent is a quorum sensing agent, or antagonist. In another aspect, the species of microorganism is a bacterium found in mammalian oral cavities, including but not limited to Streptococcus mutens. The method can further comprise culturing at least two species of microorganisms in an interacting microhabitat array whereby at least one biofilm is formed.

The IMA can also be used to screen drug candidates that are required to contact or pass through a biofilm for therapeutic relief. For example, biofilms are known to form in the oral cavity and in the respiratory tract. For an inhaled respiratory drug to have efficacy, often it must pass through the biofilm to reach the tissue that is targeted. The ability of the drug to penetrate a biofilm may be tested in vitro by creating a model biofilm in an IMA and contacting it with the drug of interest under conditions that simulate the physiological environment.

Drug screening for antimicrobial agents is also envisioned. The IMA more accurately models the heterogenous microbial populations found in the natural environment than prior uniform chemostat systems. Thus, screening antimicrobial agents to identify activity against a biofilm formed in an IMA would be an improvement over current drug screening methods.

Also useful is a method which shows how to implement the principles of directed evolution to evolve and discover drug resistance mechanisms and resistance conferring molecules from a presently susceptible microorganism. This would allow for predicting the time of clinical efficacy of a drug prior to its wide-spread clinical use, and the discovery and characterization of the molecule that eventually confers resistance to the target microorganism. This resistance conferring molecule would also be useful for drug screening for subsequent generation of agents for future clinical use.

### EXAMPLES

### Example 1

Selection for a Variant That Efficiently Produces Molecular H₂

There exist two classes of microorganisms which use light to produce oxygen: photosynthetic bacteria such as Rhodopseudomonas viridis and the cyanobacteria such as single cell cyanobacterium Synechocystis sp. PCC 6803 or the single cell algae Chlamydomonas reinhardtii. These microorganisms are single celled and can generate H₂. Of the two microorganisms, Synechocystis sp. PCC 6803 is of the greatest interest for directed evolution because it is the first photosynthetic organism to be completely sequenced and this is a huge advantage for any genomic work. However, Chlamydomonas reinhardtii is also interesting because it is unicellular, grows quickly, forms colonies on plates and is easy to transform.

The IMA was used to direct the evolution of cyanobacteria to optimize H₂ gas production.

Arrays of microhabitats offer a fundamental advantage in that the small volume and consequently small numbers of bacteria in each space allow for fluctuations in the genotype within a microhabitat to compete efficiently with the established colony, avoiding the tyranny of numbers if a mutated strain would have to compete with a vast number of dominant bacteria. The “optimum” volume of each microhabitat for accelerating evolution and selection is an easily determined parameter. The volume is varied in conjunction with both theoretical analysis and empirical results.

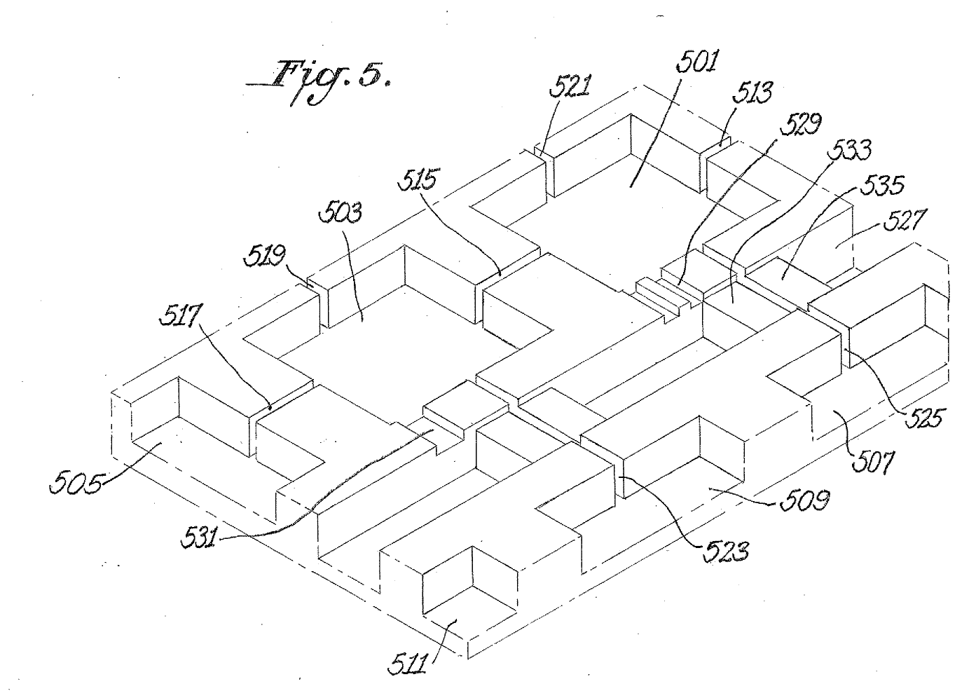

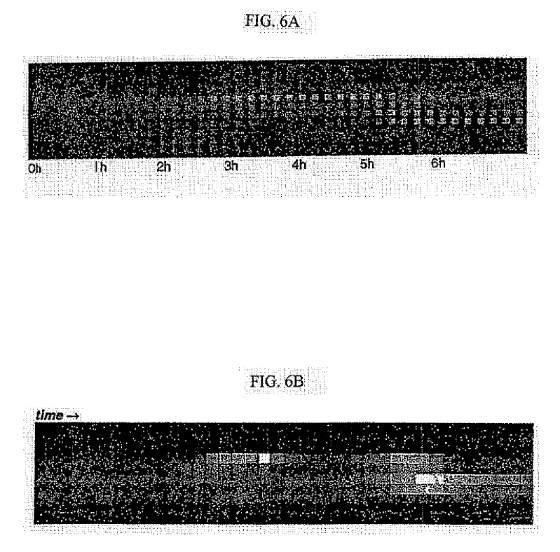

An IMA according to FIG. 1 and having 0.1 micron deep nanofeed channels was used to study cyanobacteria populations. Synechocystis sp. PCC 6803 was engineered to express Green Fluorescent Protein. The IMA was inoculated with Synechocystis in growth medium at a very low density of ten bacteria per microhabitat. The population dynamics were astonishingly complex in that bacteria moved between chambers in both wave-like and chaotic manner. See FIG. 6.

The reward/punishment cycle may be performed by using a scanning mirror technology. The scanning mirrors consist of a very fast (less than 1 millisecond settling time) servo-controlled mirror, which may be coupled to an acousto-optic optical filter which can in less than 1 microsecond select a wavelength and set an intensity from an all-lines argon-krypton laser. Those colonies that produce H₂ at a high rate may be rewarded with more light at the wavelength where H₂ photoproduction is highest, while those colonies that are poor H₂ producers are both punished and urged to evolve: we may starved them for actinic light and increase mutagenic UV light (at a wavelength of <350 nm) to increase the mutation rate. Other suitable mutagenic agents include X-rays, gamma rays, and mutagenic chemical agents. Since the microhabitats are interconnected, rapidly growing colonies that are good H₂ producers are allowed to gain more space and are rewarded with more light.

The devices and methods of the disclosure may be used to manipulate a dynamic landscape of opportunity microhabitats. Chambers under UV radiation correspond to “destroyed microhabitats” (state −1) and UV free chambers correspond to “suitable microhabitats” (state 0). See FIG. 7, Microhabitat dynamics are easily modulated by applying the lethal action of UV laser beams upon microorganisms. Adding a chamber to a laser “hit list” drives a local population (located at x belonging to the set L) to extinction by habitat destruction:
1ₓ→−1ₓ [1]
Habitat destruction can also affect empty patches. Thus:
0ₓ→−1ₓ [2]
By removing a chamber from the laser's “hit list” we can “create” a suitable habitat patch:
−1ₓ→0ₓ [3]
which later can be colonized by propagates coming from populations in near-by microhabitats. Unlike colonization-extinction cycles of the microorganisms in the culture, these new chamber reactions are generated by laser operations (which are under our control). With these laser operations we implement a regime of landscape dynamics which guide the microorganism to evolve in ways we desire.

In another embodiment, an alternative reward is used: The delivery of nutrient fluid for good H₂ producers. The delivery of fluids to specific chambers is implemented in the 1-D array by fabricating a separate fluid channel for each microhabitat. In one aspect, the transverse 0.1 micron deep channels have strictly laminar flow profiles which confine fluids within single chambers across the width. The connection channels are long enough (100's of microns) that cross-diffusion is not a problem on the hour time scale. In an alternative embodiment, microvalves control access of fluid to each particular microhabitat, using the methods, for example, of Balagadde F K, You L, Hansen C L, Arnold F H, Quake S R (2005) Science 309:137-140.

Poorly performing cells may be destroyed by lethal UV radiation. Other types of radiation, e.g. gamma- or X-rays, can be used. In another variation, cells in poorly functioning microhabitats are punished via either starvation or the delivery of toxins or mutagenic chemicals by fluid channels that supply a single microhabitat.

Remote detection of the local H₂ gas concentration may be achieved by use of an enzyme developed by the microorganisms. The soluble hydrogenase of the aerobic proteobacterium Ralstonia eutropha catalyzes the reduction of the colorimetric redox dye benyzl viologen (BV). When the hydrogenase is present in the input fluid flow to the microhabitat or is immobilized on the surface, the hydrogenase, in the presence of H₂ gas, catalyzes the oxidation of H₂ and reduces colorless redox dye benzyl viologen (BV₂₊) to the red light absorbing BV+ [3]. A camera, in this case a three color chip CCD camera, imaging the array, with each pixel, or alternatively, group of pixels, mapped to each microhabitat may be used to determine the level of BV+ being produced by the cyanobacteria in each microhabitat and hence the H₂ production of that particular microstrain. Decisions of reward and punishment for a population in a given microhabitat may be made based on the combination of growth rate and H₂ production.

In an alternative embodiment the H₂ assay is confined to a fluid channel (e.g. 107). In this version, effluent from a microhabitat is mixed into the fluid channel and the redox reaction occurs there. This embodiment minimizes interference of the cyanobacteria with the assay.

One of skill in the art can further redesign the present chips based on this disclosure in order to compartmentalize the bacterial microcolonies.

### Example 2

H₂ Production by Chlamydomonas reinhardtii

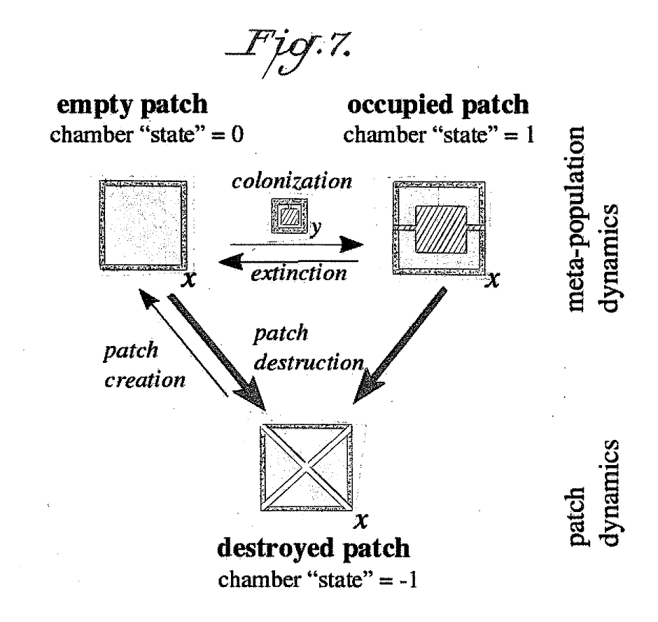

Chlamydomonas reinhardtii single-celled motile algae, was introduced into a variant of the IMA of Example 1. Because Chlamydomonas are larger than E. coli or cyanobacteria, modifications to the IMA disclosed in FIGS. 1 and 2 were needed. Microhabitats of 1000 microns by 1000 microns (top view) by 160 microns deep were connected by 500 micron-long corridors. The microhabitats were arranged in an “undulating” pattern rather than strictly linear. See FIG. 8. The device, however, is a 1D device. Dual fluid channels flanked the microhabitats and were optionally connected to the microhabitats by nanoslits that prevented escape of the Chlamydomonas. The microhabitats, which in this embodiment were squares, constitute a microincubator, each of which has small corridors of diameter 10 microns or less which allow microorganisms in adjacent microhabitats to exchange with each other in a slow way.

There are several unique features of this device. 1) Because only a few thousand rather than many 1000s of cells inhabit each microhabitat, it is far easier for genetic fluctuations to compete with the dominant species. Since one aim is the rapid and directed synthesis of H₂ it is important that mutations that produce higher levels of hydrogen gas be quickly identified and rewarded, even if they are slower growing or have other non-competitive properties, 2) The microhabitats are optically thin (on the order of 20 microns to 400 microns thick) so that light can uniformly illuminate the entire depth of the microhabitat. 3) The transparent nature of the microhabitat allows us to make sensitive colorimetric measurements of H₂ gas remotely in each microhabitat, as discussed below. 4) Each microhabitat is “weakly coupled” via a 10 micron-wide channel with neighboring habitats, so that bad performing sub-species in a microhabitat can be punished and good performing bacteria can be allowed to move into microhabitats where the species is weak, as part of the reward process.

C. reinhardtii photosynthetically synthesize O₂ under normal conditions. The algae are capable of photosynthetically producing H₂ and can produce H₂ in the dark and under certain anaerobic conditions. Interestingly, C. reinhardtii are haploid and divide by mitosis with a generation time of about eight hours. An additional advantage of Chlamydomonas as a test microorganism is that the genome has been sequenced.

After growing in the IMA, some but not all wells produced gas. See FIG. 8B.

The generation of H₂ in a microfabricated chip can be monitored with use of a sensor such as a sensor having a sensitivity down to 4 nanomoles of hydrogen.

The ability to separately generate H₂ and O₂ on a microfabricated chip may permit development of a nano/microfabricated fuel cell.

### Example 3

Nutrient Limitation

To make an ecosystem with a rate-limited supply of resources, we weakly linked microhabitats to two feeder channels for the supply of food. See FIGS. 1-2. In this rate-limited scenario, microorganisms must adapt their demands on their environment. Each of the two feeder channels was connected to the microhabitats via five nanoslits that were only 0.2 microns deep but 15 microns wide and 20 microns long. Thus, they acted as weak links between the microhabitat and the feeder channels. These nanoslits allow nutrients (and waste) to diffuse into and out of the microhabitats but are too thin for E. coli to pass through. The nanoslits provide a critical role beyond the supply of food and removal of waste. By building a different number (m) of nanoslits feeding different microhabitats, we introduced relative niche differences among collections of microhabitats. Cf. FIG. 1 and FIG. 3. Coupling between a microhabitat and a feeder channel is denoted by λ. We can build microhabitats with no exchange, λₘᵢₙ=0; intermediate exchanges, m×λ* for m belonging to the set (1, . . . , 9); and maximum exchange, λₘₐₓ=10×λ*. The value λ* here represents the contribution to the exchange rate by a single nanoslit. In this way, adaptive (fitness) landscapes can be created by patterning ecotopes (spatially connected collections of microhabitats sharing the same λᵢ) onto the habitat spatial distribution The 1D experiments disclosed here were conducted in three types of adaptive landscapes: (i) a flat one, consisting of a single ecotope of microhabitats where all 10 nanoslits are open; (ii) a black & white landscape, consisting of two ecotopes (at the right of the array we place microhabitats with all 10 nanoslits open, and on the left we put microhabitats with all nanoslits closed); and (iii) a more complex “rugged” landscape, consisting of three zones to the left a nutrient-limited “stress” domain made of microhabitats with all nanoslits closed (no supply), at the center a high nutrient-supply zone, separating the stress zone from a rugged zone (to the right), in which the rugged zone is made of clusters of all-open and partially open microhabitats embedded on a desert of stressed microhabitats.

The growth of populations and the growth and movement of metapopulations was complex in that populations arose and died or moved to nearby but not necessarily immediately adjacent microhabitats.

### Example 4

A One-Dimensional Array

We constructed a one-dimensional (1D) array of coupled microhabitats; the running index i here is used to denote the ith microhabitat. The corridors “coupling” microhabitats are designed to be narrow enough so that each microhabitat can be viewed as a local niche in a much larger adaptive landscape generated by the heterogeneous array of habitat patches. There are three fundamental parameters that characterize the habitat in this array of coupled microhabitats: (i) the local carrying capacity, K (patch size), of bacteria in the Ith microhabitat; (ii) the coupling strength, J_{i,i+1} (corridor structure), between adjacent microhabitats; and (iii) the coupling strength, λ (number of nanoslits), between the microhabitat and feeding channels that allow food to diffuse into, and waste out of, a given microhabitat.

In general, vectors K, J, and λ (landscape parameters) can be made a strong function of the index i, so that nanoscale patchy environments can be designed to test the fitness of microorganisms to different ecotopes of the landscape. We have addressed the question of how bacterial metapopulations behave when allowed to populate such landscapes.

FIG. 9A shows the dynamics of an E. coli population in a single microhabitat (zero-dimensional device) with all nanoslits open (λₘₐₓ). Although initially the density of bacteria follows a pattern of exponential growth, the weak coupling to an external resource (via λ) results in some unusual behavior: oscillations occur at least at two distinctive frequencies (high and low).

A simple analysis of diffusion through the nanoslit to the microhabitat gives us an expression for the contribution of a single nanoslit to the exchange rate between the microhabitat and the feeding channels:
λ*˜D_{w}×A/(I×V) [4]
where A is the nanoslit's total cross-sectional area, V is the microhabitat's volume, I is the length of the nanoslit, and D_{w}, is the average diffusion coefficient of resources and waste. From the volume of a microhabitat (V=3×10⁵ micron²), the approximate diffusion constant of small molecules such as amino acids (D_{w}˜10⁻⁵ cm²s⁻¹), the area of a 0.2 micron-deep and 20 micron-long nanoslit (i.e., 4 micron²), and the width of the nanoslit (I=15 micron), we found that λ*˜10⁻³·s⁻¹.

The population density ρ(t) of an microhabitat can be modeled by the logistic equation:
(1/ρ)dρ/dt=r(w)×(1−ρ/K) [5]

Here, the per capita growth rate (dρ/dt) is determined by two factors: space and resources. Space limitation is represented as the logistic (1−ρ/K) environmental resistance, where the parameter K represents the carrying capacity of the microhabitat. Resource-based growth rate r(w) is a function of habitat quality 0≦w≦1 inside the microhabitat but relative to the concentration of resources in the feeding channels. Without these resources, cells cannot grow. Thus, following resource competition theory, we use
r(w)=w×[1/Tᵣ]−[1/Tₘ] 6]
as our resource utilization function. Tilman D (1982) Resource Competition and Community Structure (Princeton Univ Press, Princeton). Here, 1/Tₘ represents the rate of cell death and 1/Tᵣ represents the birth rate achieved when the medium inside the microhabitat is fresh LB (w=1). After the biomass of the cells starts growing and transforming the medium, w decreases. Waste-saturated medium means w=0.

The feeder channels supply the microhabitat with fresh LB by λ-limited diffusion into (and waste out of) it through its nanoslits. The rate dw/dt at which resource quality changes inside the microhabitat is then the difference between inward diffusion and consumption by E. coli, normalized by the efficiency s by which resources are converted into bacterial biomass. Thus,
d/dt(w)=λ×(1−w)−ε×w×[1/Tᵣ]ρ [7]
where ε is the price of turning nutrient into biomass, i.e. a microorganism. From the known volume of a microhabitat (V) and the approximate volume of a single E. coli (0.5 micron³) we can estimate (as an upper limit) that a close-packed microhabitat has an average carrying capacity=10⁶ E. coli cells. In practice, however, a microhabitat typically saturates at about K*˜10⁴ E. coli cells.

When our microecosystem is in a regime of resource limitation, the picture goes like this; as food resources are depleted, Eqns. 6 and 7 predict that the growth rate w/Tᵣ will become less than the death rate 1/Tₘ and the population in the microhabitat will start going extinct. However, resources can diffuse in from the feeder channels and growth can reinitiate; this can give rise to oscillations in the population density due to the diffusional lag between consumption and supply. Although high-frequency spikes and lower-frequency bumps exist, our model cannot accommodate both at the same time. Only a single frequency basic oscillation for population density vs. time is expected from our model. For a fixed environment λ, the frequency of the oscillation is determined by an microorganism's life-history strategy [ε, Tₙ Tₘ]. A consortium of phenotypes would be expected to exhibit more frequencies.

### Example 5

Selection Paradigms

It is important to emphasize that the growth and evolution of even bacterial species is a complex process because of the highly evolved biological networks in bacteria which enable bacteria to compete or cooperate with other strains for limited resources. We are guiding the evolution of the competing species, and are, of necessity, working within the framework of competing species in stationary growth conditions.

The Prisoner's Dilemma is widely used as a metaphor for the evolution of cooperation. In the standard formal form the game, the prisoner's dilemma goes like this: There are two players which can choose (independently but simultaneously) to cooperate (C), or defect (D), in any one encounter. If both players cooperate, they get a payoff of magnitude R (a “reward”); if one defects and the other cooperates, D gets the games' biggest payoff T (the “temptation”), while C gets the smallest, S. If both defect, both get P. The payoff matrix can be written as

$$( \begin{matrix}R & S \\ T & P\end{matrix} ) . \qquad [ 8 ]$$

The prisoner's dilemma occurs when elements of the payoff matrix (eqn. [5]) satisfy the following inequality,
T>R>P>S. [9]
The paradox embedded in the prisoner's dilemma relationship (eqn. [6]) is that strategy D is unbeatable at any one round of the game. At the same time, if both players play the game iteratively, both end up with less total payoff than if had they cooperated. This paradox is a problem not just for humans but also for E coli and cyanobacteria evolving in the IMA structures. As resources change during growth and cell density increases, the microorganisms change their gene expression and enter what is called the GASP phase. GASP is an acronym for the growth advantage in stationary phase (GASP) phenotype.

Bacterial genetics is used to implement the iterative (and spatial) prisoners dilemma of bacterial societies on our metapopulations biochips. UV laser disturbance is used to study the game (and its evolutionary strategies) in different dynamic landscapes of opportunity patches.

E. coli (much like us) are victims of the tragedy of the commons. In closed cultured systems (i.e., test tubes) after mid-log phase, resources become limiting and the amount of toxic waste builds. At this stage, cells signal to one another (i.e. by secreting auto-inducer molecules like Al2). The result is cooperation among the cells and a cessation of replication. This is the onset of “stationary phase” , which is a standard condition in the IMA during processes to enhance mutation, positive selection, and negative selection (“evolving”) of the microorganisms. Stationary phase E. coli cells express the gene rpoS and produce sigma factor us (a genetic switch) responsible for the relevant gene regulation needed to express stationary phase programs.

Under this “phenotypic state”, wild-type organisms “cooperate” (by not dividing) in order to save resources. After a while in stationary phase, the GASP mutants appear in the population. Thus, when we look carefully, we must notice that stationary phase is really very dynamic. Constant replacements of ever more “GASP” mutants occur. The older we let the culture get, the more mutants appears up to a point when diversity starts accumulating and highly diverse and polymorphic assemblages start to develop. This is the onset of “stationary phase” in the IMA.

GASP mutants in general carry mutations on the rpoS gene. In particular the GASP phenotype of the early mutants that take over stationary phase cultures of wild-type cells is thought to arise due to the presence of the rpoS₈₁₉ allele. The rpoS₈₁₉ allele has a 46 base pair duplication of part of the original sequence of the wild-type allele (rpoS_{wt}) which allowed easy identification of PCR fragments by running agarose gels. We used primers 5′-GTTAACGACCATTCTCG-3′ and 5′-TCACCCGTGCGTGnC-3′ to amplify the section of the rpoS gene containing the difference in sequence between the moS_{wt} and _{rpoS19}alleles. PCR was performed and products of the rpoS gene were separated by electrophoresis on an agarose gel for identification.

### Example 6

E. coli Metapopulation Dynamics in a Flat Landscape.

Equivalent microhabitats in an interactive 1D array are termed a flat landscape. The spatial dynamics of E. coli growing on a flat landscape where all microhabitats have all their nanoslits open (λᵢ=λₘₐₓ˜10λ*, Vi) are not necessarily uniform in either space or time. See FIG. 9. FIG. 9A consists of a time-ordered stack of epifluorescence images of all 85 microhabitats in this array. The array was scanned every 10 min for 300 times, sampling a total of 3,000 min (Δt=2.1 days). Each row of images of the 85 microhabitat array represents the configuration of the array at time t. Local (microhabitat) population density average ρᵢ, at any given time t can then be calculated by integrating epifluorescence intensity for all pixels within the ith microhabitat.

The dynamics of the landscape average p(t)=Σρ/85 resemble what is seen in batch cultures. In particular, after a lag period of ˜400 min during which little growth occurs in the array, a period of growth (the exponential phase) followed by landscape saturation (stationary phase) at 10⁴ cells per microhabitat (K*˜3×10¹⁰ per ml) was observed. In FIG. 9B the beginning and end of the exponential phase are demarcated by vertical dashed lines. Because the landscape is flat, we would expect that over time the bacteria would inhabit all of the microhabitats. However, because coupling is weak (small J_{i, i+1}), a metapopulation emerges. Thus, whereas the population density of an individual microhabitat (local scale, shown in FIG. 9B as a solid curve) shows sharp rises and falls in density, occupancy of the entire array (landscape scale) shows a much slower growth rate and smoother dynamics than the single microhabitat (FIG. 9B). The fit (dotted curve) of the logistic map to the globally averaged occupancy/microhabitat (dashed curve) shown in FIG. 9B yields a Tᵣ˜250 min (˜4 h). The reason for this slow growth is that there are localized E. coli populations distributed over the landscape, interacting through local extinction and colonization processes operating at multiple spatial and temporal scales. In particular, although the density seems constant (stationary phase) at the global scale, at the local microhabitat scale there are clear dynamics. On the other hand, although the global averages are at exponential phase because of continuous range expansion, individual local populations can be in stationary or in death phasesln FIG. 9A Right, we show a (mesoscale) 15-microhabitat-wide, “parent” population giving “birth” to a new population spreading to the right and settling six microhabitats away. Thus, in a flat habitat landscape, E. coli aggregates its biomass at multiple scales satisfying a careful balance between vacancy and occupancy. These multiscale aggregates correspond to spatial versions of the classical phases of growth: lag, log, stationary, and death. Zooming into a particular microhabitat, we can observe pulses of exponential growth with a 10- to 20-min time constant (local colonization events), stationary-phase, and death-phase oscillations (local extinction events) occurring at multiple spatial and temporal scales Unlike the zero dimensions case, in one dimension the bacteria can migrate into nearby microhabitats, so growth can continue in a delayed fashion throughout the microhabitat array.

### Example 7

Population Dynamics

The methods and devices disclosed here are useful for the study of colonies of microorganisms in microfabricated spaces. The methods and devices of this disclosure are designed to mimic the complex world that microorganisms inhabit in the real world as opposed to an agar plate in terms of how bacteria colonize and communicate with each other. We found, as was anticipated by many biological studies, of course, that even E. coli had a complex but understandable signaling pathway which under conditions of metabolic stress drove the microorganisms to form a microcolony in the smallest enclosed volume available. The fact that we could simulate this behavior mathematically using a set of physically realistic coupled set of equations governing bacterial density and chemotactic response gave us faith in the general power of this approach.

We also studied the behavior of both E. coli and V. harveyi in these structures and were able to observe the clustering of the bacteria together into compact colonies as they responded to metabolic stress. Under metabolic stress the E. coli cluster into small inner squares.

E. coli and V. harveyi Accumulation and Quorum Sensing.

In particular, epifluorescence images were obtained of green fluorescent protein (GFP) labeled E coli in M9 minimal media as they accumulate into a central 250 micron by 250 micron enclosure via a 40 micron wide channel through 100 micron-wide walls. After 3 hours the density of cells was more than seven times greater inside than outside. Also, dark-field images were obtained of V. harveyi after 8 hours in a maze having the narrowest passages 100 micron wide. V. harveyi formed clusters of dense population. These areas corresponded to areas of intrinsic luminescence as seen by photon counting, indicating active quorum sensing in areas where the cells have accumulated at high density.

GFP-Expressing E. coli Growing in 100 Micron×100 Micron Microhabitats

E. coli expressing green fluorescent protein was expressed in this array. Each microhabitat is a voxel 100 microns×100 microns×20 microns, and is linked to its neighbors by a 10 micron wide channel. The 2D IMA is a simple design in which nutrients are feed into one end of the array and waste is removed at the other end, with a syringe pump delivering carefully metered amounts of fluid. The basic design had a layout of 6 arrays, with each array containing 85 microhabitats. E. coli grow vigorously in these devices, as can be seen by epifluorescence images of small sections of an array 12 hours after the array was inoculated with a low density of GFP expressing E. coli. For example, in this case the bacteria have reached stationary levels of population density. We have also shown that pulsed UV light at 337 nm from a N₂ laser can be used to kill bacteria within an individual array.

### Example 8

Screening for Agents

The IMAs disclosed above are suitable for screening for agents that modulate microbial activity, particularly those that modulate the stationary phase, or for screening for agents that affect biofilms. The IMA design can also be advantageously modified to provide a separate fluid channel for each microhabitat, such as on one side. In the latter design, different agents or a control vehicle can be administered in separate fluid channels. A 2D IMA such as illustrated in FIG. 5 is readily adapted to multiple fluid channels such that an agent can be administered to a row of microhabitats. Alternatively, screening is performed by administering a gradient of agent, or a series of agents spaced in one fluid channel using an IMA design such as in FIG. 1 or in FIG. 5.

The agents suitable for evaluation as modulators of bacterial stationary phase or bacterial biofilms is not limited and can be any agent suspected of activity, such as the agents disclosed in US2006/0228384, US2006/0052425, US2006/0229259, US2006/0228965, US2006/0165648, US2005/0215772, US2004/0147592, US2003/0105072, US2002/0177715, and US2001/0021398.

It will be obvious that the present methods may be varied in many ways. Such variations are not to be regarded as a departure from the spirit and scope of the methods, and all such modifications as would be obvious to one skilled in the art are intended to be included within the scope of the following claims. The breadth and scope of the present invention is therefore limited only by the scope of the appended claims and their equivalents. All of the references and patent publications referred to herein are incorporated herein by reference in their entireties.

We claim:

1. An interacting microhabitat array for microorganisms comprising a plurality of microhabitats in a substrate wherein at least two microhabitats are connected in series by at least one corridor, wherein the corridor is of sufficient size to allow the microorganism to move between microhabitats.

2. The interacting microhabitat array of claim 1, further comprising at least one fluid supply channel.

3. The interacting microhabitat array of claim 1 wherein the size of the corridor restricts but does not completely obstruct movement of the microorganism.

4. The interacting microhabitat array of claim 2 wherein the channel is connected to the microhabitat by at least one nanoslit, wherein the nanoslit is of a size such that the microorganism cannot pass through it.

5. The interacting microhabitat array of claim 1 further comprising a detector in one or more microhabitats that is capable of detecting a change in the activity of the microorganism.

6. The interacting microhabitat array of claim 1 wherein at least one microhabitat is between about 20 to about 2000 microns in top width.

7. The interacting microhabitat array of claim 1 wherein at least one microhabitat is between about 6 and about 320 microns in side width.

8. The interacting microhabitat array of claim 1 wherein at least ten microhabitats are connected in series.

9. The interacting microhabitat array of claim 1 further comprising microorganisms.

10. The interacting microhabitat array of claim 9 wherein the microorganism is a virus, mycoplasma, Bacterium, Archea, or Protist, Fungi or other Eukaryote, or combinations thereof.

11. The interacting microhabitat array of claim 1 wherein the substrate comprises silicon, derivatized silicon, silicon having embedded enamel or bone, or combinations thereof.

12. A method of evaluating population colonizations and extinctions in a metapopulation of microorganisms comprising introducing at least one species of microorganism into an interacting microhabitat array wherein at least two microhabitats are connected in series by at least one corridor, wherein the corridor is of sufficient size to allow the microorganism to move between microhabitats, providing nutrients to the microhabitats, removing waste from the microhabitats, and detecting population changes during a plurality of generations across the interacting microhabitat array.

13. The method of claim 12 wherein the microorganism is capable of expressing a fluorescent protein.

14. A method of screening for an agent capable of modulating biofilms comprising: a) culturing a species of microorganism capable of forming a biofilm in the interacting microhabitat array of claim 1 such that at least one biofilm is formed, (b) adding a test agent to the biofilm, and (c) detecting whether a change occurs in the biofilm in the presence of the test agent.

15. The method of claim 14 wherein the change detected is selected from an increase in the amount of the species of microorganism forming the biofilm, a decrease in the amount of the species of microorganism forming the biofilm, an increase in the ratio of planktonic cells to cells in the biofilm, a decrease in the ratio of planktonic cells to cells in the biofilm, an increase in the size of the biofilm, or a decrease in the size of the biofilm.

16. The method of claim 14 wherein the agent is a quorum sensing agent.

17. The method of claim 14 wherein the species is a bacterium found in mammalian oral cavities.
